(19) United States
(12) Patent Application Publication
(10) Pub. No.: US 2026/0148845 A1
(43) Pub. Date: May 28, 2026

(54) MEDICAL DATA INTEGRATION AND ANALYSIS SYSTEM

(71) Applicant: China Medical University, Taichung City (TW)

(72) Inventors: Der-Yang Cho, Taichung City (TW); Wen-Sheng Feng, Hsinchu County (TW); Wei-Cheng Chen, Taichung City (TW); How-Yang Tseng, Taichung City (TW); Jiun-Yi Lin, Keelung City (TW); Ching-Yao Chou, Nantou County (TW); Hsin-Mei Chen, Taichung City (TW)

(73) Assignee: China Medical University, Taichung City (TW)

(21) Appl. No.: 19/240,227

(22) Filed: Jun. 17, 2025

Related U.S. Application Data
(60) Provisional application No. 63/725,027, filed on Nov. 26, 2024.

(51) Int. Cl. G16H 40/40; G16H 10/60

(52) U.S. Cl. CPC G16H 40/40; G16H 10/60

(57) ABSTRACT

A medical data integration and analysis system includes a plurality of medical devices, a medical record database, a data flow processing device, a database managing device, an artificial intelligence predicting device and a vision displaying device. The medical devices respectively provide a plurality of detection data flows. The medical record database provides a record data flow for a plurality of patients. The data flow processing device is configured to assign the detection data flows and the record data flow. The database managing device for storing the detection data flows and the record data flow. The artificial intelligence predicting device provides a predicting data flow including the predicting results to the data flow processing device. The vision displaying device is for displaying the detected features, the patient features and the predicting results.

### RELATED APPLICATIONS

This application claims priority to U.S. Provisional Application Ser. No. 63/725,027, filed Nov. 26, 2024, which is herein incorporated by reference.

### BACKGROUND

### Technical Field

The present disclosure relates to a data integration and analysis system. More particularly, the present disclosure relates to a medical data integration and analysis system.

### Description of Related Art

Recently, since Internet of Things (IoT) and Artificial Intelligence Technique grow quickly, Artificial Intelligence of Things (AIoT) which combines both are generated to achieve better data management and analysis effect.

By the advent of an aging society, AIoT can be used in medical care. Till now, over 62% of medical devices are wearable or implantable for home monitoring, and the remaining 38% are in Intensive Care Units (ICUs), areas with less focus on AI application.

In hospitals, critical medical devices, including patient monitors, ventilators, infusion pumps, and hemodynamics monitors, are deployed in ICU settings, generating huge data including patient reactions, patient history medical records, laboratory analysis results and medical images. Moreover, multidisciplinary healthcare professionals such as intensivists, cardiac surgeons, respiratory therapists, nurses, pharmacists and nutritionists have to work together in the ICUs. However, there is an inadequacy for the conventional system to integrate medical data and manage the patients. The conventional system only has the ability to process the single type of electronic medical record (EMR) or the data of the medical devices, and is lack of the ability to integrate data from multiple sources, leading to scatted patient information, and it is hard for the critical care team to obtain the whole status of the patient in real time. Furthermore, conventional alerting systems depend on single data source usually, and cannot provide accurate prediction and real time alerts by combining multiple data sources; consequently, the emergency status may not be reacted in real time, and the medical risk is increased. In addition, the alerts are scatted in each system, lacking centralized management and unified presentation. Therefore, it is hard for the critical care team to integrate all the key data as there is a requirement to react quickly.

Therefore, how to increase the connection and the data integration of the medical devices in the medical center such as ICUs and for the medical team to quickly obtain the status of the patients becomes a target those in the field purse.

### SUMMARY

According to one aspect of the present disclosure, a medical data integration and analysis system includes a plurality of medical devices, a medical record database, a data flow processing device, a database managing device, an artificial intelligence predicting device and a vision displaying device. The medical devices respectively provide a plurality of detection data flows. The medical record database provides a record data flow for a plurality of patients. The data flow processing device is configured to assign the detection data flows and the record data flow. The data flow processing device includes a plurality of receiving queues and a plurality of replication queues. The data flow processing device selects a data flow key of each of the detection data flows and the record data flow according to a data flow key selecting strategy. A remainder is obtained by dividing each of the data flow keys by a number of the receiving queues, the detection data flows and the record data flow are assigned to the receiving queues according to the remainders, and the replication queues replicate the receiving queues. The database managing device is signally connected to the data flow processing device for storing the detection data flows and the record data flow, the database managing device sets a plurality of data table primary keys, and the data table primary keys are respectively identical to the data flow keys. The artificial intelligence predicting device is signally connected to the data flow processing device and the database managing device. The artificial intelligence predicting device obtains a plurality of detected features of each of the detection data flows and a plurality of patient features of each of the patients of the record data flow to provide a predicting result of each of the patients, and the artificial intelligence predicting device provides a predicting data flow including the predicting results to the data flow processing device. The vision displaying device is signally connected to the data flow processing device and the database managing device for displaying the detected features, the patient features and the predicting results.

According to one aspect of the present disclosure, a medical data integration and analysis system includes a plurality of medical devices, a medical record database, a data flow processing device, a database managing device, an artificial intelligence predicting device and a vision displaying device. The medical devices respectively provide a plurality of detection data flows. The medical record database provides a record data flow for a plurality of patients. The data flow processing device is configured to assign the detection data flows and the record data flow. The data flow processing device includes a plurality of receiving queues and a plurality of replication queues. The data flow processing device selects a data flow key of each of the detection data flows and the record data flow according to a data flow key selecting strategy. A remainder is obtained by dividing each of the data flow keys by a number of the receiving queues, the detection data flows and the record data flow are assigned to the receiving queues according to the remainders, and the replication queues replicate the receiving queues. The database managing device is signally connected to the data flow processing device for storing the detection data flows and the record data flow, the database managing device sets a plurality of data table primary keys, and the data table primary keys are respectively identical to the data flow keys. The artificial intelligence predicting device is signally connected to the database managing device. The artificial intelligence predicting device obtains a plurality of detected features of each of the detection data flows and a plurality of patient features of each of the patients of the record data flow to provide a predicting result of each of the patients, and the predicting results are stored by the database managing device. The vision displaying device is signally connected to the data flow processing device and the database managing device for displaying the detected features, the patient features and the predicting results and provides an alert.

### BRIEF DESCRIPTION OF THE DRAWINGS

The present disclosure can be more fully understood by reading the following detailed description of the embodiment, with reference made to the accompanying drawings as follows:

FIG. 1 shows a block diagram of a medical data integration and analysis system according to one embodiment of the present disclosure.

FIG. 2 shows a curve of a low tidal volume ventilation of the medical data integration and analysis system of the embodiment of FIG. 1.

FIG. 3 shows curves of a P/F ratio and FiO₂ of the medical data integration and analysis system of the embodiment of FIG. 1.

### DETAILED DESCRIPTION

The embodiments of the present disclosure will be illustrated with drawings hereinafter. In order to clearly describe the content, many practical details will be mentioned with the description hereinafter. However, it will be understood by the reader that the practical details will not limit the present disclosure. In other words, in some embodiment of the present disclosure, the practical details are not necessary. Additionally, in order to simplify the drawings, some conventional structures and elements will be illustrated in the drawings in a simple way; the repeated elements may be labeled by the same or similar reference numerals.

In addition, the terms first, second, third, etc., are used herein to describe various elements or components, these elements or components should not be limited by these terms. Consequently, a first element or component discussed below could be termed a second element or component. Moreover, the combinations of the elements, the components, the mechanisms and the modules are not well-known, ordinary or conventional combinations, and whether the combinations can be easily completed by the one skilled in the art cannot be judged based on whether the elements, the components, the mechanisms or the module themselves are well-known, ordinary or conventional.

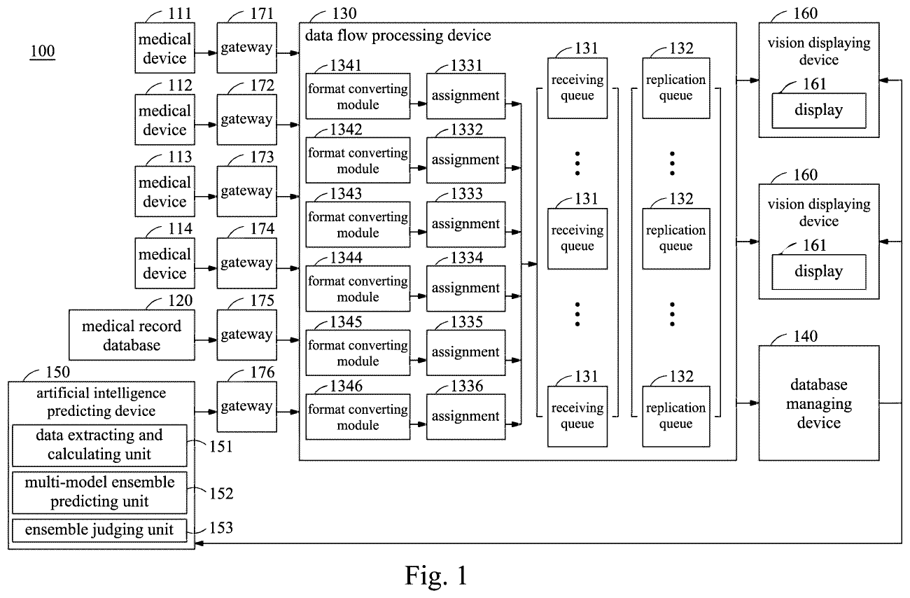

FIG. 1 shows a block diagram of a medical data integration and analysis system 100 according to one embodiment of the present disclosure. The medical data integration and analysis system 100 includes a plurality of medical devices 111, 112, 113, 114, a medical record database 120, a data flow processing device 130, a database managing device 140, an artificial intelligence predicting device 150 and a vision displaying device 160.

The medical devices 111, 112, 113, 114 respectively provide a plurality of detection data flow. The medical record database 120 provides a record data flow for a plurality of patients. The data flow processing device 130 is configured to assign the detection data flows and the record data flow. The data flow processing device 130 includes a plurality of receiving queues 131 and a plurality of replication queues 132. The data flow processing device 130 selects a data flow key of each of the detection data flows and the record data flow according to a data flow key selecting strategy. A remainder is obtained by dividing each of the data flow keys by a number of the receiving queues 131, the detection data flows and the record data flow are assigned to the receiving queues 131 according to the remainders, and the replication queues 132 replicate the receiving queues 131. The database managing device 140 is signally connected to the data flow processing device 130 for storing the detection data flows and the record data flow, the database managing device 140 sets a plurality of data table primary keys, and the data table primary keys are respectively identical to the data flow keys.

The artificial intelligence predicting device 150 is signally connected to the data flow processing device 130 and the database managing device 140. The artificial intelligence predicting device 150 obtains a plurality of detected features of each of the detection data flows and a plurality of patient features of each of the patients of the record data flow to provide a predicting result of each of the patients, and the predicting results are stored by the database managing device 140. The vision displaying device 160 is signally connected to the data flow processing device 130 and the database managing device 140 for displaying the detected features, the patient features and the predicting results.

Therefore, with that the data flow processing device 130 may receive the detection data flows and the record data flow, and with that the data flow keys may be selected according to the data flow key selecting strategy, the efficiency for assigning the detection data flows and the record data flow may be increased. In addition, with that the artificial intelligence predicting device 150 may predict the status of the patients and that the vision displaying device 160 may display all the data, the medical team is easy to obtain the status of the patients.

Precisely, the medical devices 111, 112, 113, 114 may be devices which can monitor the physiological indexes of the patients and transmit monitor data, such as patient monitors, ventilators, infusion pumps, and hemodynamics monitors. In the health facility, a number of the medical devices 111, 112, 113, 114 may be multiple, and some of them may be disposed at the same space to detect the status of the same patient. In the present embodiment, the medical devices 111, 112 may be disposed at a first ICU to detect the status of one patient, and the medical devices 113, 114 may be disposed at a second ICU to detect the status of another patient. The medical devices 111, 113 may for example be patient monitors to detect features such as a heart rate, a blood pressure, a respiratory rate, a blood oxygen saturation (SpO₂) and so one. The medical devices 112, 114 may for example be ventilators for detecting a tidal volume, an exhalation minute volume, an inspired fraction of oxygen (FiO₂) and so on. In other embodiments, a number and types of the medical devices are not limited. The medical devices may for example be infusion pumps, hemodynamics monitors and so on, and the hemodynamics monitor provide a cardiac output (CO).

The medical record database 120 may include a fixed or removable random access memory (RAM), a read-only memory (ROM), a flash memory, hard disk drive (HDD), a solid state drive (SSD) or similar element or the combination thereof. The medical record database 120 may obtain the electronic medical record (EMR) system including outpatient records, emergency records, admission notes, medicine records, inspection records, physician notes and so on via an application programming interface (API), and therefore the medical record database 120 includes the historical medical records of the patient, The medical record database 120 may further receive data from the picture archiving and communication system (PACS), and the present disclosure is not limited thereto.

The data flow processing device 130 may for example be a central processing unit (CPU) or other programmable devices such as a micro control unit (MCU), a microprocessor, a digital signal processor (DSP), a programmable logic controller (PLC), an application specific integrated circuit (ASIC), a graphics processing unit (GPU), an arithmetic logic unit (ALU), a complex programmable logic device (CPLD), a field programmable gate array (FPGA) and other similar devices or the combination thereof. In the present embodiment, the data flow processing device 130 may receive the data first, and then the data is stored into the database managing device 140. Therefore, the artificial intelligence predicting device 150 may provide a predicting data flow including the predicting results to the data flow processing device 130, and then the predicting results can be stored in the database managing device 140, but the present disclosure is not limited thereto.

The medical data integration and analysis system 100 may further includes a plurality of gateways 171, 172, 173, 174, 175, 176 respectively signally connected to the medical devices 111, 112, 113, 114, the medical record database 120 and the artificial intelligence predicting device 150 for transmitting the detection data flows, the record data flow and the predicting data flow to the data flow processing device 130. In the present embodiment, a number of the gateways 171, 172, 173, 174, 175, 176 is six, and the gateways 171, 172, 173, 174, 175, 176 may be connected to the medical devices 111, 112, 113, 114, the medical record database 120 and the artificial intelligence predicting device 150 via a wire or wireless connection, such as a RS-232 port, Bluetooth or a wireless network. The gateways 171, 172, 173, 174, 175, 176 may transmit to the data flow processing device 130 via the wireless network.

In the data flow processing device 130, each of the medical devices 111, 112, 113, 114 corresponds to different topic. The detection data flow of each of the medical devices 111, 113 corresponds to a topic of “Topic_PM”, the detection data flow of each of the medical devices 112, 114 corresponds to a topic of “Topic_VEN”, the record data flow of the medical record database 120 corresponds to a topic of “Topic_HIS”, and the predicting data flow of the artificial intelligence predicting device 150 corresponds to a topic of “Topic_AI”, but the present disclosure is not limited thereto. The detection data flows corresponding to the same topic but from different medical devices 111, 112, 113, 114 may be assigned to different receiving queues 131. The detection data flow may include a patient identifier (ID), a device ID, a timestamp, and the measured value, and any one or the combination of the patient ID, the device ID and the timestamp may be suitable for being served as the data flow key. In the present embodiment, the data flow key selecting strategy is preferably to select the patient ID to be served as the data flow key, and the device ID is the second choice. The record data flow may include the patient ID, a table ID and a table content. The table ID may correspond to a specific column or row of the table, and the table content is the content corresponds to the specific column or row. The data flow key selecting strategy chooses the combination of the patient ID and the table ID to be served as the data flow key. Similarly, the predicting data flow may include the patient ID, the table ID and the table content. As the artificial intelligence predicting device 150 provides the predicting data flows to the data flow processing device 130, the data flow key selecting strategy chooses the combination of the patient ID and the table ID to be served as the data flow key which can be divided by the number to obtain the remainder, thereby assigning the predicting data flows.

The data flow processing device 130 may further include a plurality of assignments 1331, 1332, 1333, 1334, 1335, 1336, respectively signally connected to the gateways 171, 172, 173, 174, 175, 176. The assignments 1331, 1332, 1333, 1334, 1335, 1336 may be respectively used for assigning the detection data flows, the record data flow and the predicting data flow. For example, a number of the receiving queues 131 may be n and a number of the replication queues 132 is also n, that is, a first receiving queue 131 to an nth receiving queue 131 and a first replication queue 132 to an nth replication queue 132 being included. The binary codes of the data flow keys may be divided by n by the assignments 1331, 1332, 1333, 1334, 1335, 1336 to obtain remainders r, the data flows may be delivered to the r+1 receiving queue 131, and at least one of the first replication queue 132 to the nth replication queue 132, e.g., the r+1 replication queue 132, replicates the r+1 receiving queue 131. Therefore, the data flow may be stored in both the receiving queues 131 and the replication queues 132, thereby ensuring the safety and the integrity of the data. In addition, if one node may include a specific amount of receiving queues and replication queues, the data flow processing device may connect a plurality of nodes in serial, and a number of the nodes may be 1 to 5, e.g., 3, to ensure the system stability. The redundancy storing strategy highly increases the fault tolerance and the data usability, preventing data loss caused by single point of failure. Hence, as connecting a huge number of devise, e.g., 60 thousand devices, the efficiency is stable, and the stability is increased to 99.9999%, which highly increases the efficiency of data management and search, and satisfies the requirement of connecting a huge number of devices and dealing with the data in real time.

In the embodiment, the data flow processing device 130 may further include a plurality of format converting modules 1341, 1342, 1343, 1344, 1345, 1346 for converting formats of the detection data flows. The original formats of the medical devices 111, 112, 113, 114 may be different and may for example be Health Level Seven International (HL7) v2 or JavaScript Object Notation (JSON). Hence, with the format converting modules 1341, 1342, 1343, 1344, 1345, 1346, the original format may be transferred to a unique format, and the unique format may be, but not be limited to, Fast Healthcare Interoperability Resources (FHIR) standard. It is noted that, in the embodiment, the format converting modules 1341, 1342, 1343, 1344, 1345, 1346 are connected to the gateways 171, 172, 173, 174, 175, 176 one by one by setting the internet protocol (IP) address, but the present disclosure is not limited thereto.

The database managing device 140 may include a fixed or removable random access memory (RAM), a read-only memory (ROM), a flash memory, hard disk drive (HDD), a solid state drive (SSD) or similar elements or the combination thereof. The database managing device 140 may read the receiving queues 131 and the replication queues 132 of the data flow processing device 130 to read and store data flows, thereby ensuring the data may be efficiently managed and searched as required to satisfy the daily operation requirement of the medical team. With invoking the reading function of the data flow processing device 130, the data flows may be stored in specific addresses according to different receiving queues 131 and the replication queues 132. Hence, the detected features of the patient such as a heart rate, a blood pressure, a respiratory rate, a blood oxygen saturation, a tidal volume, an exhalation minute volume, an inspired fraction of oxygen and a stroke volume (SV) provided by the medical devices 111, 112, 113, 114, the patient features such as a height, a sex and blood gas analysis results, such as a partial pressure of oxygen (PO₂) and a partial pressure of carbon dioxide (PCO₂) provided by the medical record database 120 may be stored in the database managing device 140. Moreover, with setting the data table primary keys of the database managing device 140 to be respectively identical to the data flow keys, the indexes are established by the database managing device 140. Furthermore, for the receiving queues 131 and the replication queues 132 belonging to the detection data flows of the medical devices 111, 112, 113, 114, timeline indexes may be also established, thereby facilitating data searching and increasing the query efficiency.

The artificial intelligence predicting device 150 may include a data extracting and calculating unit 151, a multi-model ensemble predicting unit 152 and an ensemble judging unit 153. The data extracting and calculating unit 151 is configured to obtain a plurality of selected members of the detected features and the patient features of each of the patients from the database managing device 140, and to calculate at least one parameter based on at least two of the selected members. The multi-model ensemble predicting unit 152 includes a plurality of models, and each of the models provides a classification result according to the selected members and the at least one parameter of each of the patients. The ensemble judging unit 153 decides the predicting result from the classification results of the models of each of the patients based on an ensemble learning algorithm.

The data extracting and calculating unit 151 may obtain the detected features and patient features of each patient from the database managing device 140, and the obtained detected features and patient features are defined as the selected members. For example, the selected members may include a height, a sex, a partial pressure of carbon dioxide, a tidal volume and an exhalation minute volume. The height and the sex are used to calculate a parameter as a predictive body weight (PBW). The predictive body weight, the partial pressure of carbon dioxide, the tidal volume and the exhalation minute volume may be used to calculate a parameter as a ventilatory ratio of condition (1), and parameters as a P/F ratio of condition (2) and a low tidal volume ventilation of condition (3) may be further calculated.

$$VR = \frac{V e \times {P}_{CO 2} \times V t}{100 \times 37.5 \times P B W} . \qquad ( 1 )$$

$$PF = {P}_{O 2} / {FI}_{O 2} . \qquad ( 2 )$$

$$LTVV = Vt / PBW . \qquad ( 3 )$$

VR represents the ventilatory ratio, Ve represents the exhalation minute volume, P_{CO2} represents the partial pressure of carbon dioxide, Vt represents the tidal volume, PBW represents the predictive body weight, PF represents the P/F ratio, P_{O2} represents the partial pressure of oxygen, FL_{O2} represents the inspired fraction of oxygen, and LTVV represents the low tidal volume ventilation.

The models of multi-model ensemble predicting unit 152 may be a support vector machine (SVM) model, a multiple-layer perceptron (MLP) model and an extreme gradient boosting (XGB) model. Each model may be trained by relative data, and can provide its own classification result based on the selected members and the parameters obtained by the data extracting and calculating unit 151. The classification result may be whether the patient suffers the acute respiratory distress syndrome (ARDS).

The artificial intelligence predicting device 150 may also use the XGB model to predict the risk of antibiotics resistance and sepsis onset and may analyze the patient medical history, the antibiotics usage, and the data from hematology analyzers. A long short-term memory (LSTM) model may also be used to analyze the electrocardiogram (EKG) data for identify the likelihood of an ST-elevation myocardial infarction (STEMI) heart attack. Therefore, a plurality of models can be established in the artificial intelligence predicting device 150, and the user may choose suitable models based on the prediction content.

The ensemble learning algorithm of the ensemble judging unit 153 may for example be voting, averaging, boosting, bagging/bootstrap aggregation or stacking. In the embodiment, majority voting of voting may be used, and the result accounting for the majority of the classification results is defined as the predicting result.

The vision displaying device 160 may be electronic devices such as computers and tablets. A number of the vision displaying devices 160 may be two and be disposed at two different spaces. Each vision displaying device 160 may include a display 161 for showing different data. Mufti-level frame configuration may be used and include a main frame, a personal frame and a detailed frame. The main frame displays the unit statistics, the infection controlling map, the negative pressure isolation ward area data, the contact isolation ward area data and the regular ward area data. The unit statistics may include the bed quantity, the bed capacity (occupied/available), list of physicians, the ARDS number and ratio, average of acute physiology and chronic health evaluation II (APACHE II) score, the relative mortality rates, the number and usage rate of the major medical devices 111, 112, 113, 114, the diagnostic rate of major diseases and the using rate of bundles. In one embodiment, the display 161 may display the bed map, a personal frame may be shown as pointing a specific bed, and the personal frame includes the data of the patient corresponding to the specific bed, such as the bed number, the name, the sex, the main disease, APACHE II, the date for staying in the ICU, the isolation status, the physician and nurse belonging thereto, the used medical devices 111, 112, 113, 114, the real time detecting result (detected features), the drug data such as dose, starting time and ending time, the obtained parameters and the predicting result from the artificial intelligence predicting device 150, focusing on the health data of each patient. Moreover, the medical record data and the alerting data may be respectively shown according to the six systems of ICU care such as the respiratory system, the cardiovascular system and so on. Three-dimension body map may further be used for showing the physiological state and the usage of the medical devices 111, 112, 113, 114. As a result, the medical providers can understand and analyze the patient's status intuitively to quickly obtain the patient's real time status.

The detailed frame may provide details and traceable data illustration functions for specific medical topics or medical departments. Interactive statistic charts may be used to show historical data of a specific medical index, detail data of specific medical information, and real time bedside images, thereby assisting the medical team to conduct deep analysis and dynamic monitors to provide accurate diagnosis and treatment decisions.

In addition, if the data monitored by the medical devices 111, 112, 113, 114 are abnormal, except for alerting by the medical devices 111, 112, 113, 114 themselves, the vision displaying device 160 may also provide an alert to remind relative personals. The alert may be a sound or a changing of the text, and the alerting signal position may be shown in the three-dimension body map. Moreover, as the predicting result of the artificial intelligence predicting device 150 shows that there is a high risk of suffering disease or being abnormal, the vision displaying device 160 may also provide an alert to ask the relative personals to deal with the issues.

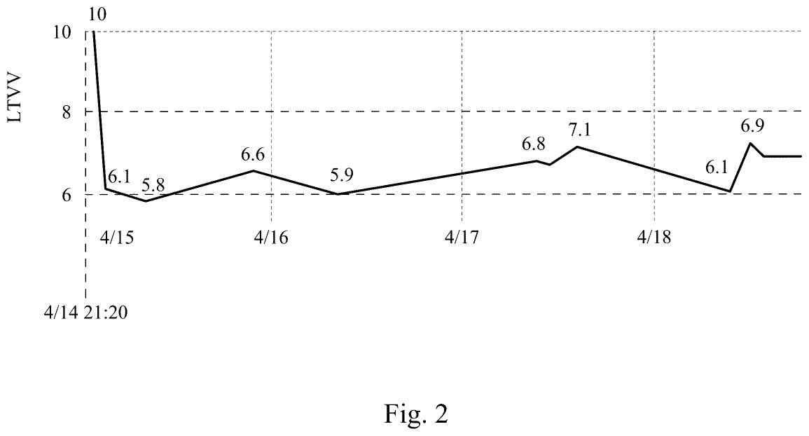

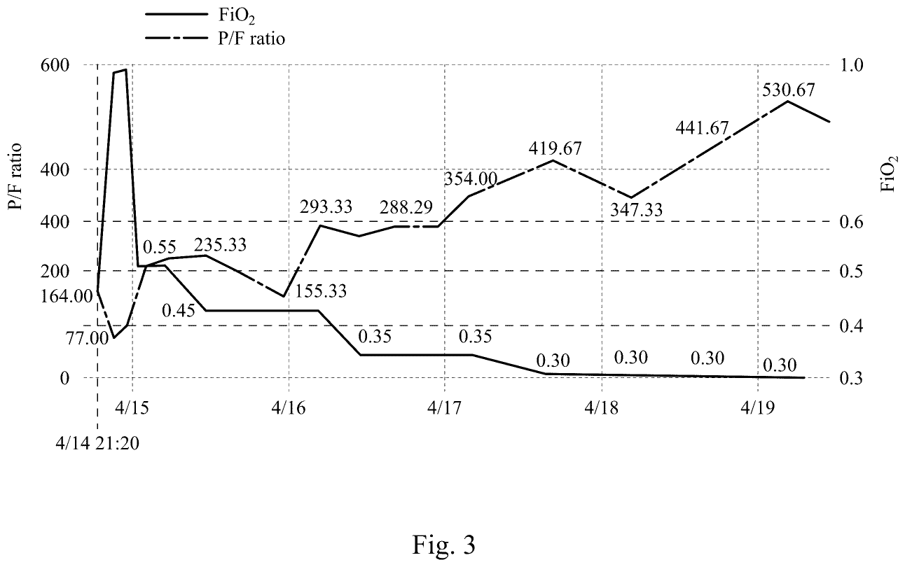

FIG. 2 shows a curve of the low tidal volume ventilation of the medical data integration and analysis system 100 of the embodiment of FIG. 1. FIG. 3 shows curves of the P/F ratio and FiO₂ of the medical data integration and analysis system 100 of the embodiment of FIG. 1. As shown in FIGS. 2 and 3, at 21:20 of 4/13, the low tidal volume ventilation of a patient is 10 and is higher than the safe value, 8, the P/F ratio is 164 and is lowered than the safe value, 300, and the FiO₂ is measured to be larger than 1 and is higher than the safe value 0.4. The artificial intelligence predicting device 150 judges that the patient suffers ARDS, and the vision displaying device 160 provides an alert. After the medical team receives the alert and gives corresponding treatments such as giving antibiotics and antivirals, the patient's condition has significant improvement. At 3:30 of 4/15, LTVV is lower to 6.1 and staying in a safe range, and the P/F ratio and FiO₂ also fall into the safe range. In a statistic result, the medical data integration and analysis system 100 can increase the diagnosis and control of ARDS, the ARDS diagnosis rate is increased from 52.2% to 93.3%, and the decreased mortality is decreased from 56.5% to 39.5%.

The medical data integration and analysis system 100 may further include a remote medical device to support the medical team to conduct cooperation work in different positions. Via remote illness monitors, consultations and real time discussions, the covering area of the medical service is increased to ensure the immediacy and continuity of treatment. The remote medical device may include two levels. The first level is to support the specialists to conduct remote consultations, facilitating the real time cooperation between different specialists. The second level is to extent the view of the medical team. With integrating the monitor system, the medical team can handle the patient's status in real time, and the efficiency of collaboration care can be increased.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A medical data integration and analysis system, comprising:
a plurality of medical devices respectively providing a plurality of detection data flows;
a medical record database providing a record data flow for a plurality of patients;
a data flow processing device configured to assign the detection data flows and the record data flow, the data flow processing device comprising a plurality of receiving queues and a plurality of replication queues, the data flow processing device selecting a data flow key of each of the detection data flows and the record data flow according to a data flow key selecting strategy, wherein a remainder is obtained by dividing each of the data flow keys by a number of the receiving queues, the detection data flows and the record data flow are assigned to the receiving queues according to the remainders, and the replication queues replicate the receiving queues;
a database managing device signally connected to the data flow processing device for storing the detection data flows and the record data flow, the database managing device setting a plurality of data table primary keys, wherein the data table primary keys are respectively identical to the data flow keys;
an artificial intelligence predicting device signally connected to the data flow processing device and the database managing device, the artificial intelligence predicting device obtaining a plurality of detected features of each of the detection data flows and a plurality of patient features of each of the patients of the record data flow to provide a predicting result of each of the patients, the artificial intelligence predicting device providing a predicting data flow comprising the predicting results to the data flow processing device; and
a vision displaying device signally connected to the data flow processing device and the database managing device for displaying the detected features, the patient features and the predicting results.

2. The medical data integration and analysis system of claim 1, wherein the data flow processing device further comprises a plurality of format converting modules for respectively converting formats of the detection data flows.

3. The medical data integration and analysis system of claim 1, further comprising a plurality of gateways respectively signally connected to the medical devices, the medical record database and the artificial intelligence predicting device for transmitting the detection data flows, the record data flow and the predicting data flow to the data flow processing device.

4. The medical data integration and analysis system of claim 3, wherein the data flow processing device further comprises a plurality of assignments respectively signally connected to the gateways.

5. The medical data integration and analysis system of claim 1, wherein the artificial intelligence predicting device comprises:
a data extracting and calculating unit configured to obtain a plurality of selected members of the detected features and the patient features of each of the patients from the database managing device, and to calculate at least one parameter based on at least two of the selected members;
a multi-model ensemble predicting unit comprising a plurality of models, each of the models providing a classification result according to the selected members and the at least one parameter of each of the patients; and
an ensemble judging unit deciding the predicting result from the classification results of the models of each of the patients based on an ensemble learning algorithm.

6. The medical data integration and analysis system of claim 5, wherein the models are a support vector machine model, a multiple-layer perceptron model and an extreme gradient boosting model.

7. A medical data integration and analysis system, comprising:
a plurality of medical devices respectively providing a plurality of detection data flows;
a medical record database providing a record data flow for a plurality of patients;
a data flow processing device configured to assign the detection data flows and the record data flow, the data flow processing device comprising a plurality of receiving queues and a plurality of replication queues, the data flow processing device selecting a data flow key of each of the detection data flows and the record data flow according to a data flow key selecting strategy, wherein a remainder is obtained by dividing each of the data flow keys by a number of the receiving queues, the detection data flows and the record data flow are assigned to the receiving queues according to the remainders, and the replication queues replicate the receiving queues;
a database managing device signally connected to the data flow processing device for storing the detection data flows and the record data flow, the database managing device setting a plurality of data table primary keys, wherein the data table primary keys are respectively identical to the data flow keys;
an artificial intelligence predicting device signally connected to the database managing device, the artificial intelligence predicting device obtaining a plurality of detected features of each of the detection data flows and a plurality of patient features of each of the patients of the record data flow to provide a predicting result of each of the patients, the predicting results being stored by the database managing device; and
a vision displaying device signally connected to the data flow processing device and the database managing device for displaying the detected features, the patient features and the predicting results and providing an alert.

8. The medical data integration and analysis system of claim 7, wherein the artificial intelligence predicting device comprises:
a data extracting and calculating unit configured to obtain a plurality of selected members of the detected features and the patient features of each of the patients from the database managing device, and to calculate at least one parameter based on at least two of the selected members;
a multi-model ensemble predicting unit comprising a plurality of models, each of the models providing a classification result according to the selected members and the at least one parameter of each of the patients; and
an ensemble judging unit deciding the predicting result from the classification results of the models of each of the patients based on an ensemble learning algorithm.

9. The medical data integration and analysis system of claim 8, wherein the models are a support vector machine model, a multiple-layer perceptron model and an extreme gradient boosting model.

10. The medical data integration and analysis system of claim 8, wherein the selected members comprise a height, a sex, a partial pressure of carbon dioxide, a tidal volume and an exhalation minute volume.
